(19) United States
(12) Patent Application Publication
(10) Pub. No.: US 2022/0241521 A1
(43) Pub. Date: Aug. 4, 2022

(54) DEVICE FOR FIBRIN-BIOPOLYMER-FORMING SUBSTANCE APPLICATION

(71) Applicant: Freedom Corp., Los Angeles, CA (US)

(72) Inventors: Rodrigo Guedes Nunes, Sao Paulo (BR); Renata Lima Lopes, Fortaleza (BR)

(21) Appl. No.: 17/165,503

(22) Filed: Feb. 2, 2021

(51) Int. Cl. A61M 11/02; A61K 9/12; A61K 38/36; B05B 11/00; A61K 38/48

(52) U.S. Cl. CPC A61M 11/02; A61K 9/124; A61K 38/482; A61K 38/363; B05B 11/0062; A61K 9/122

(57) ABSTRACT

The two devices described allow for greater control over application of a fibrin biopolymer and yet remain simple enough to be used by both medical personnel and by non-medical workers. One of the devices includes chambers where components necessary for formation of a fibrin biopolymer are stored and are pushed by plungers connected to an actuator upon the actuator receiving pressure from the user, with the components mixing in a chamber within the device and flowing out of a tube connected to the mixing chamber before formation of the fibrin biopolymer is completed. The other device dispenses formulations that include components necessary for formations of a fibrin biopolymer onto a skin of a patient using one or more propellants, with the formation of the fibrin biopolymer being initiated after the formulations are dispensed on the patient's skin and are intentionally mixed together.

### FIELD

This application relates in general to medicine, and in particular, to a device for fibrin-biopolymer-forming substance application.

### BACKGROUND

Biopolymers are polymers that are composed of naturally-occurring substances, such as amino acids, sugars, lipids, and nucleic acids. While many biopolymers are produced naturally in living organisms, they can also be synthesized outside of living organisms, and depending on their properties and ease of synthesis, have been shown to have potential in many areas of medicine, including damaged tissue treatment, drug delivery, and gene therapy.

One particularly promising class of biopolymers are fibrin biopolymers that are the most abundant component of blood clots. Naturally, fibrin biopolymers are formed from fibrinogen molecules (with fibrinogen being a glycoprotein that circulates in the blood of all invertebrates) being enzymatically processed by thrombin or thrombin-related enzymes upon the organism sustaining a vascular injury that requires clotting. Despite the promise of exogenous fibrin biopolymers for wound treatment, their application remains largely limited to clinical settings and out of reach of every day consumers that buy medical products over the counter. An example of such limitation is seen in International Application Publication No. WO2015172215, published Nov. 19, 2015, entitled “Fibrin Sealant For Topical Use, Method For Producing Same and Use Thereof,” by Rui Seabra Ferreira Junior, Benedito Barravieira, and Silvia Regina Sartori Barravieira (“the Ferreira publication”), the disclosure of which is incorporated by reference. The Ferreira publication discloses combining fibrinogen of animal origin with a thrombin-like serinoprotease (known as gyroxine) purified from venom of a snake Crotalus durissus terrificus in the presence of calcium chloride, with the serinoprotease cleaving the fibrinogen molecules into fibrin monomers. The fibrin monomers polymerize in the presence of calcium into a fibrin biopolymer, which has sealing and adhesive properties, and can be used in wound care. The Ferreira publication states that the three components necessary to create the fibrin biopolymer (the fibrinogen, the serinoprotease, and a diluent that includes calcium chloride) are kept in separate vials at −20° C. prior to being combined using multiple syringes and topically applied to a skin wound. The requirement for −20° C. storage and the use of multiple syringes for creation and application of the fibrin biopolymer makes this technology useful only within a hospital or a doctor's office setting, preventing the technology's application for treatment of every day skin wounds.

Attempts have been made to make the fibrin biopolymer technology more accessible to non-medical personnel, but such attempts have turned up deficient. For example, International Patent Application Publication No. WO2018191800, entitled “Device For Applying Fibrin Biopolymers,” published on Oct. 25, 2018, by Moacyr Ramos Bighetti and Ana Silvia Sartori Barraviera Seabra Ferreira, the disclosure of which is incorporated by reference, discloses an adhesive bandage whose support structure is impregnated with powdered serinoprotease purified from snake venom and a fibrinogen-rich cryoprecipitate extracted from large animals. The bandage further includes a compartment positioned above the support structure that includes a diluent liquid, which when released onto the support structure causes a formation of a sealant and transparent film on the bandage that can be applied to a wound. The application of such a bandage is limited by the size and shape of the bandage, thus being able to cover only small wounds of a particular shape. Further, if such a bandage is applied to a wound larger than the area of the bandage, the adhesive portions of the bandage would come into contact with the wound surface and damage the wound surface when removed, thus aggravating the wound and interfering with the healing process. Finally, as such the fibrin biopolymer tends to solidify within seconds of the mixing of necessary components, if a user fails to place the bandage onto a correct spot within those seconds, the bandage becomes useless.

Finally, International Patent Application Publication No. WO2018191801, entitled “Method for Producing A Fibrin Biopolymer, Means For Applying Said Fibrin Biopolymer and Method For Applying Said Fibrin,” published Oct. 25, 2018, by Moacyr Ramos Bighetti and Ana Silvia Sartori Barraviera Seabra Ferreira (“the Bighetti publication”), the disclosure of which is incorporated by reference, discloses a spray bottle that has three compartments, with one compartment storing a powdered fibrinogen-rich cryoprecipitate extracted from large animals, another compartment storing a serinoprotease supplied from snake venom, and the third compartment storing a liquid diluent. Each compartment includes an inert gas under a high pressure, which acts as a propellant to drive the contents of each compartment through a spray nozzle. The Bigghetti publication states that the expelled contents of the compartments mix with each other in the air on their path to the wound to be treated and on the wound to form a biopolymer. However, unless the spray bottle is used at a particularly short distance (which would allow to cover only a small patch of a patient's skin with the fibrin biopolymer), the powdered compartments expelled from the bottle would start dispersing from the path on which the mixing would occur, thus both reducing the amount of fibrinogen and serinoprotease available for forming the fibrin polymer and resulting in the landing of the powders on an undesired surface, where the unreacted powders can be broken down as nutrients for bacteria and other pathogens. Further, the diluent (with the powdered components mixed in), can splatter, and in addition to gravity influencing the direction at which the diluent moves before polymerization is complete, leads to difficulty in controlling the shape of the resulting fibrin biopolymer film that results from the application of the spray.

Accordingly, there is a need for a device that allows to easily control an application of a fibrin biopolymer to a wound surface without wasting biopolymer components and that can be used in both medical personnel and as an over-the-counter product.

### SUMMARY

The two devices described below allow for greater control over application of a fibrin biopolymer and yet remain simple enough to be used by both medical personnel and by non-medical workers who buy the devices over the counter. One of the devices includes chambers where components necessary for formation of a fibrin biopolymer are stored and are pushed by plungers connected to an actuator upon the actuator receiving pressure from the user, with the components mixing in a chamber within the device and flowing out of a tube connected to the mixing chamber before formation of the fibrin biopolymer is completed. The other device dispenses formulations that include components necessary for formations of a fibrin biopolymer onto a skin of a patient using one or more propellants, with the formation of the fibrin biopolymer being initiated after the formulations are dispensed on the patient's skin and are intentionally mixed together by a patient or a person providing care to the patient. The formulations are more viscous than water, and thus are not easily moved from their positions before they are intentionally mixed together, providing control over the start of the formation of the fibrin biopolymer that is necessary when the biopolymer needs to be applied to a large portion of the patient's skin. Further, both devices allow to reduce the waste of components present in earlier works.

In one embodiment, a fibrin biopolymer formation and application device is provided. The device includes a housing and a plurality of compartments formed within the housing, the plurality of compartments including: a fibrinogen compartment whose contents include fibrinogen; a serinoprotease compartment whose contents comprise a serinoprotease, wherein the serinoprotease cleaves the fibrinogen into fibrin monomers when combined with the fibrinogen; and a diluent compartment whose contents comprise a diluent, the diluent comprising a cofactor, wherein the fibrin monomers polymerize into a fibrin polymer in the presence of the cofactor. The device further includes the mixing chamber formed adjacent to the compartments, the mixing chamber including an opening; a tube positioned at one end of the housing and interfaced to the opening in the mixing chamber; and an actuator positioned at another end of the housing and connected to a plurality of plungers, wherein a pressure applied on the actuator drives the plungers to push at least a portion of the contents of all of the compartments into the mixing chamber to form a mixture in which the serinoprotease cleaves the fibrinogen into the fibrin monomers and the polymerization begins and wherein at least a portion of the mixture flows out via the opening into the tube and flows out from a distal end of the tube prior to a completion of the polymerization of the fibrin monomers in the at least the portion of the mixture into the fibrin biopolymer.

In a further embodiment, a device for fibrin-biopolymer-forming substance application is provided. The device includes a housing and a plurality of compartments formed within the housing, contents of each of the compartments including one or more propellants, the plurality of the compartments including: a fibrinogen compartment whose contents further include a formulation more viscous than water and comprising fibrinogen; a serinoprotease compartment whose contents further include a further formulation more viscous than water and comprising a serinoprotease, wherein the serinoprotease cleaves the fibrinogen into fibrin monomers when combined with the fibrinogen; and a cofactor compartment whose contents include an additional formulation more viscous than water and include a cofactor, wherein the fibrin monomers polymerize into a fibrin biopolymer in the presence of the cofactor. The device further includes a plurality of valves, each of the valves integrated into one of the compartments and adapted to take a closed position and an open position; a plurality of at least partially hollow conduits, each of the conduits including a first end that is at least partially interfaced to one of the valves, the first end including a first opening, each of the conduits further including a second end including a second opening that is interfaced to one of a plurality of orifices within an actuator; the actuator interfaced to all of the conduits and configured to apply pressure to all of the valves via the conduits upon being pressed by a user, wherein the pressure shifts the valves from the closed position into the open position and exposes the first opening of each of the conduits to the contents of the compartments, and wherein at least part of the contents of each of the compartments is propelled by the one or more propellants via the first openings of the conduits to flow via the conduits out of the second openings and out of the orifices of the actuator while the valves remain in the open position, and wherein the fibrinogen formulation, the serinoprotease formulation, and the cofactor formulation form the fibrin biopolymer when mixed outside of the actuator.

Still other embodiments will become readily apparent to those skilled in the art from the following detailed description, wherein are described embodiments by way of illustrating the best mode contemplated. As will be realized, other and different embodiments are possible and the embodiments' several details are capable of modifications in various obvious respects, all without departing from their spirit and the scope. Accordingly, the drawings and detailed description are to be regarded as illustrative in nature and not as restrictive.

### BRIEF DESCRIPTION OF THE DRAWINGS

FIG. 1 is a diagram showing a front view of a fibrin biopolymer formation and application device in accordance with one embodiment.

FIG. 2 is a diagram showing a top view of the device of FIG. 1 in accordance with one embodiment.

FIG. 3 is a diagram showing a vertical cross-section of the device of FIG. 2 at level marked E-E.

FIG. 4 shows a horizontal cross-sectional view of the device at the level marked as B-B on FIG. 1 in accordance with one embodiment.

FIG. 5 shows a horizontal cross-sectional view of the device at the level marked as C-C on FIG. 1.

FIG. 6 shows a horizontal cross-sectional view of the device at the level marked as D-D on FIG. 1 in accordance with one embodiment.

FIG. 7 is a diagram, provided for purposes of illustration, showing use of the device of FIG. 1 for covering a wound with the fibrin polymer.

FIG. 8 is diagram showing a device for fibrin-biopolymer-forming substance application in accordance with one embodiment.

FIG. 9 is a diagram showing a top view of the device of FIG. 8 in accordance with one embodiment.

FIG. 10 is a diagram showing a vertical cross-section of the device of FIG. 9 at level marked H-H when the valves are in closed positions.

FIG. 11 is a diagram showing the vertical cross-section of the device of FIG. 9 at level marked H-H after the valves have shifted into an open position.

FIG. 12 is a diagram showing an example of the formulations deposited by the device of FIG. 8 on a wound on a patient's arm.

FIG. 13 is a diagram showing an example of the fibrin biopolymer formed on top of the wound shown with reference to FIG. 12 following the mixing of the formulations applied to the wound with the device of FIG. 8.

### DETAILED DESCRIPTION

Improved control over where how and where a fibrin biopolymer is applied to wounds can be provided via two devices is described below. When applied, the fibrin polymer accelerates the rate that wounds are healed, reduces possibilities of infections, and protects the wounded area from further external impacts. Due to being simple to use, the devices described below can be sold over the counter as well used by trained medical personnel. Further, at least some embodiments of the devices can be stored at room temperature for long enough to be of practical use without significant potency degradation, further contributing to practicability of the devices.

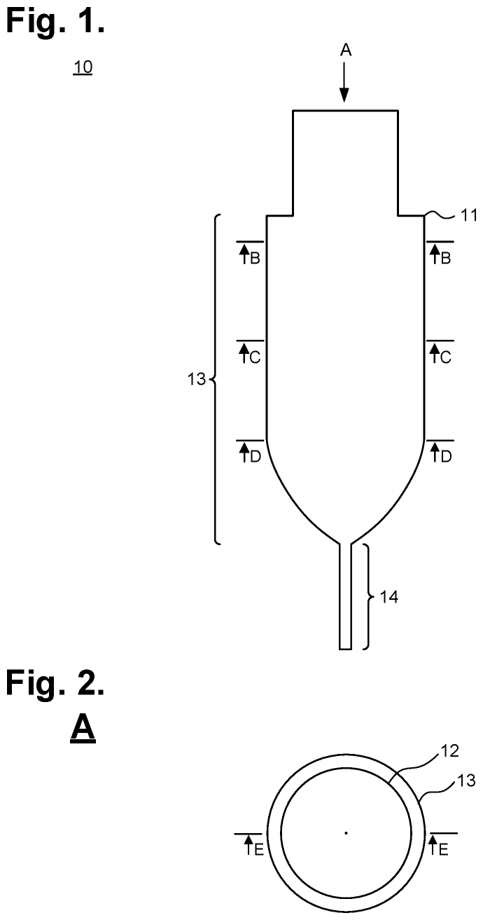

FIG. 1 is a diagram showing a front view of a fibrin biopolymer formation and application device 10 in accordance with one embodiment. Externally, the device 10 includes a housing 13 that is shaped as a conical cylinder, though other shapes are also possible. A hollow tubular appendage 14 (a “tube” from hereinafter) is attached to one end of the housing 13. In one embodiment, the tube 14 can be an integrally formed on the housing 13; in a further embodiment, the tube 14 can be removably attachable to the housing 13, such as with the tube 14 being a needle whose hub attaches to an adaptor formed on the housing. In one embodiment, where the tube 14 is removable, the tubes 14 could have the same hub diameter (thus being able to attached to the same housing 13 adaptor), but would have a different diameter at the tip through which a polymerizing mixture escapes the device 10, allowing to choose a tube most appropriate to a size of an area that needs to be covered with the biopolymer. For example, if a wound is a narrow cut, a tube with a narrower diameter at the tip could be used, while if the wound is larger, a tube 14 with a larger diameter tip could be attached to the housing 13. In a further embodiment, even if the tube 14 is removable, only the same kind of tubes 14 could be attached to the housing 13. In a still further embodiment, multiple removable tubes 14 could be used for dispensing a polymerizing mixture from the same device 10. For example, if a large amount of the polymerizing mixture needs to be dispensed by the device 10, and a first removable tube 14 gets clogged before the entirety of the polymerizing mixture is dispensed, the first tube 14 could be removed and a second removable tube 14 could be attached to the housing 13 to allow further dispensing of the polymerizing mixture. If the second tube 14 gets similarly clogged, additional tubes 14 could be similarly attached to the housing 13.

A button 12 is inserted into an opening on an end of the housing 13 opposite to the end to which the tube 14 is attached. The portion of the button 12 outside the housing 13 is substantially cylindrical, though in a further embodiment, other shapes are possible. Additionally, the button 12 can include appendages (not shown) attached to the cylindrical portion to increase the ease of handling of the button and prevent the button 12 from falling through the opening of the housing 13 into which the plunger is inserted. In a further embodiment, other shapes of the button 12 are possible. In a still further embodiment, external actuators other than the button 12 could be used to trigger the formation and application of the biopolymer described below. In a still further embodiment, a removable protective cap (not shown) could be placed on top of the button to prevent an accidental pressing of the button 12.

FIG. 2 is a diagram showing a top view of the device 10 of FIG. 1 in accordance with one embodiment. The top view is shown from the perspective marked as A in FIG. 1. As can be seen with reference to FIG. 2, the end of the housing 13 into which the button 12 is inserted and the top of the button 12 are both circular, though in a further embodiment, other shapes are also possible.

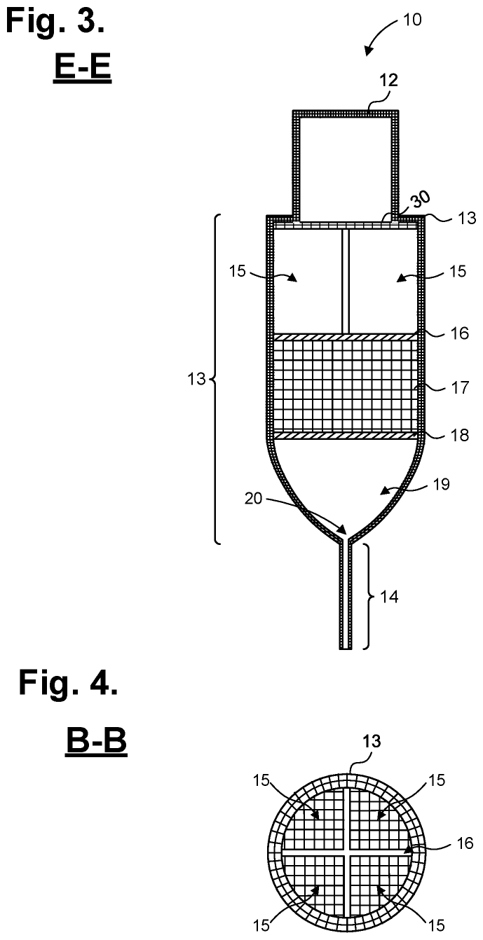

Internally, the housing 13 includes compartments where components necessary for formation of a fibrin biopolymer are stored. FIG. 3 is a diagram showing a vertical cross-section of the device 10 of FIG. 2 at level marked E-E. As can be seen with reference to FIG. 3, the portion of the button 12 within the housing 13 is connected to a base 30 that is wider than the opening within the housing 13 through which the button 12 is inserted, thus preventing the button from being pulled out from the housing 13. In the embodiment where the top portion of the housing is cylindrical, the base 13 is circular, though in a further embodiment, other shapes are also possible. All of the sides of the base 30 are in contract with the inner walls of the housing 13, thus maintaining the same orientation of the base 30 relative to the housing 13 when the base 30 moves along the length of the housing 30 due to the application of the force on the button 12.

Attached to the base 30 are a plurality of plungers 15 that rest on a seal 16 (such as a thin plastic film) separating the plungers from the compartments where components necessary to create the fibrin biopolymer are stored. In one embodiment, there are four plungers 15 attached to the base 30, as can be seen with reference to FIG. 4. FIG. 4 shows a horizontal cross-sectional view of the device 10 at the level marked as B-B on FIG. 1 in accordance with one embodiment. As can be seen with reference to FIG. 4, each of the plungers 15 can be shaped as a cylindrical sector, though in a further embodiment, in accordance to changes to the shape of the compartments 21, 23, 25, 27 (described below) or the housing 13 as a whole, other shapes would be possible. For example, if the compartments 21, 23, 25, 27 were cylindrical, the plungers 15 would also become cylindrical. In the embodiment shown with reference to FIG. 4, the plungers 15 do not touch each other, leaving a portion of the seal 16 exposed, each of the plungers 15 being positioned over one of the compartments 21, 23, 25, 27 in which the components for forming a fibrin biopolymer are stored (though separated from the compartments by the seal 16).

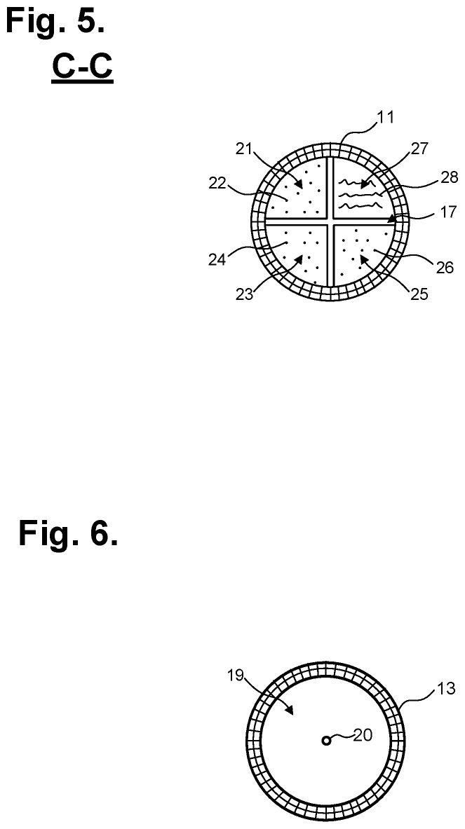

As shown with reference to FIG. 3, the compartments 21, 23, 25, 27 are defined by a partition 17 positioned between the walls of the housing 13 and are separated from the remainder of the interior of the housing by the seal 16 separating the compartments from the plungers 15 and another seal 18 (such as a thin plastic film) separating the compartments 21, 23, 25, 26 from the mixing chamber 19 described below. The arrangements of the components in the compartments is seen in detail with reference to FIG. 5. FIG. 5 shows a horizontal cross-sectional view of the device 10 at the level marked as C-C on FIG. 1. In the embodiment shown, the partition 17 is cross-shaped, defining the four compartments 21, 23, 25, and 27. In a further embodiment, if the number of the compartments 21, 23, 25, and 27 is different than 4, the shape of the partition 17 would likewise be different to create the necessary number of compartments. The compartments 21, 23, 25, and 27 are separated from each other by the partitions 17 and the seals 15, 18 tightly enough to prevent exchange of the contents of the compartments 21, 23, 25, and 27 when the device 10 is shaken or turned over. The shapes of the compartments 21, 23, 25, and 27 are complementary to the shapes of the plungers 15 located next to the compartments 21, 23, 25, and 27. Thus, if a user applies sufficient downward (relative to the orientation shown in the FIGURES) force onto the button 12, the force would be transferred through the base 30 onto the plungers 15, and cause the plungers 15 to simultaneously break through the seal 16 and enter the compartments 21, 23, 25, and 27. As the shapes of the plungers 15 and the compartments 21, 23, 25, 27 are complimentary, the plungers 15 would fit within the compartments 21, 23, 25, and 27 in the same way that a plunger fits within a barrel of a syringe. Similarly to a plunger being within a syringe, once the plungers 15 enter the compartments 21, 23, 25, 27, neither solids nor liquids nor air can escape through the ends of the compartments 21, 23, 25, 27 into which the plungers 15 are inserted. Accordingly, upon a continual application of force by the user upon the button 12, the plungers 15 would force the contents of the compartments to break through the seal 18, and enter the mixing chamber 19 described below, where the contents of the compartments would mix and begin the polymerization reaction that leads to the formation of the fibrin biopolymer. In one embodiment, if the height levels of the contents of all of the compartments is the same, the length of the plungers 15 is the same, and in that embodiment, all of the plungers 15 would break through the seal 16 and enter their respective compartments 21, 23, 25, 27 at the same time. In a further embodiment, if the height levels of the contents of each of the compartments are not the same (for example, if the volume of a diluent 28 in compartment 27 described below is greater than volume of contents of other compartments 21, 23, 25), to make sure that sufficient dissolution of the contents of the other compartments 21, 23, 25 takes place, the length of the plungers 15 are adjusted so that while the plungers 15 would not break through the seal 16 at the same time, the pressure that would apply to the contents of the compartments would cause the seal 18 under (relative to the orientation shown with reference to FIGS. 3 and 5) each of the compartments 21, 23, 25, 27 to break at the same time. Once the seal 18 is broken, the plungers 15 would travel through the entire length of the compartments 21, 23, 25, 27, thus expelling all of the contents of the compartments 21, 23, 25, and 27 into the mixing chamber 19.

The contents of the compartments 21, 23, 25, 27 are described below.

The compartment 21 (referred to as the fibrinogen compartment below) stores a powder 22 that includes fibrinogen protein molecules. In one embodiment, the powder can be a fibrinogen-rich cryoprecipitate (insoluble, cold-precipitated fraction of frozen fresh plasma). The cryoprecipitate can be extracted from large animals through techniques known in the art, such as from bovine animals (such as buffalos), though other sources of the cryoprecipitate are possible. In one embodiment, the cryoprecipitate can include in addition to the fibrinogen, factor VIII, factor V, and von Willebrand factor (such as in the case of a bubaline cryoprecipitate described in detail by the Ferreira publication cited above and whose disclosure is incorporated by reference). In a further embodiments, other components of the cryoprecipitate are possible. In a further embodiment, the powder 22 can include fibrinogen that was recombinantly produced in bacteria, yeast (or other fungi), mammalian cell culture, or other cell culture, and purified through chromatographic (such as high performance liquid chromatography or fast performance liquid chromatography) and other protein purification techniques known in the art. Once purified to a sufficient degree of purity, the fibrinogen is lyophilized to create the powder. Still other ways to produce the powder 22 that includes the fibrinogen are possible. The recombinantly produced fibrinogen can be a bovine fibrinogen (such as a buffalo fibrinogen), though other kinds of fibrinogen can also be made recombinantly. When a bovine or other non-human fibrinogen is used to make a fibrin biopolymer applied to a human, such fibrin biopolymer is considered heterologous.

The compartment 23 (referred to as the serinoprotease compartment below) stores a powder 24 that includes serinoprotease molecules capable of cleaving the fibrinogen molecules stored in the fibrinogen compartment 21 into monomers necessary for creation of a fibrin biopolymer. Such serinoprotease can be gyroxine extracted from the snake Crotalus durissus terrificus described in detail by the Ferreira publication cited above and whose disclosure is incorporated by reference. Alternatively, the gyroxine can be produced recombinantly in bacteria, yeast (or other fungi), mammalian cell culture, or other cell culture, and purified using chromatographic (such as high performance liquid chromatography or fast performance liquid chromatography) and other protein purification techniques known in the art. Once purified to a sufficient degree of purity, the gyroxine is lyophilized to create the powder. In a further embodiment, the serinoprotease (a gyroxine or a similar serinoprotease) could be derived, either through extraction or through recombinant production (as described above), from venom of other snakes, or other kinds of animals, such as when the serionoprotease is athrombin-like enzyme gyroxin B1.3 of the melon fruit fly Zeugodacus cucurbitae. Still other ways to obtain the serinoprotease are possible.

The compartment 27 (referred to as a diluent compartment below) stores a diluent 28 necessary for the serinoprotease in the powder 24 and the fibrinogen in the powder 24 to mix (upon dissolving in the diluent) and for the serinoprotease molecules to cleave the fibrinogen molecules into the fibrin monomers. Such diluent can be water, though other diluents are also possible. Within the diluent 28 are also dissolved calcium molecules, which are necessary for the fibrin monomers to polymerize into the fibrin biopolymer at a rate useful for practical applications, being a cofactor that catalyzes the polymerization reaction. The calcium molecules can be added to the diluent 28 by dissolving within the diluent 28 calcium chloride, calcium carbonate, or calcium phosphate, though dissolving other calcium-containing molecules is also possible. In a still further embodiment, another cofactor instead of calcium could be dissolved within the diluent 28. In one embodiment, the concentration of the calcium within the diluent is 20 mM to 30 mM, though other concentrations are also possible. The calcium concentration can be chosen based on the purpose of a particular device 10. For example, if a device 10 is for use in the clinical settings and is meant for producing a large amount of the fibrin biopolymer, the polymerization of the components into the fibrin biopolymer needs to be slowed down to allow the mixed components to escape from the tube before the components completely polymerize into the fibrin biopolymer and become solid. In such a case, where a patient can be expected to remain motionless when requested to prevent movement of a not-yet-polymerized mixture, the calcium concentration would be towards the 20 mM concentration or below to slow down the polymerization. On the other hand, where the device 10 is intended to be sold over-the-counter for applying a relatively small amount of the fibrin biopolymer to treat a relatively small wound, a quickly polymerizing mixture would be beneficial to make sure that the fibrin polymer covers the desired area and does not leak to undesired areas due to gravity or patient motion. In such a case, a calcium concentration close to 30 mM (or higher) to quicken the polymerization.

Finally, within the compartment 25 stores a powder 26 that includes molecules of one or more coagents that when integrated into the fibrin biopolymer promote desired objectives, such as wound healing. For example, such coagent can include an antibiotic to prevent bacterial growth under the layer of fibrin biopolymer applied to a patient's skin. Other drugs could also be coagents. For example, one or more a coagent could be an anti-cancer drug, though other kinds of drugs are also possible. Alternatively or in addition to the drug, the coagent can be a lyophilized (or otherwise in powder form) protein. For example, such coagent could be Alternagin-C (ALT-C), an ECD-disintegrin-like protein from Bothrops alternatus snake venom shows antiangiogenic activity at concentrations higher than 100 nM. As angiogenesis, formation of new blood vessels, is crucial for tumor development, adding ALT-C (or another protein from the disintegrin family) as a coagent can be used during anti-cancer treatment, as described below. Other properties of ALT-C (or other disintegrin proteins) could be utilized for other kinds of treatments, such as in wound treatment. Still other kinds of proteins are also possible as a coagent, including proteins from the lectin family, such as the lectin protein from the Aplysia dactylomela which have been shown to have potential applications in anti-cancer and wound healing treatments. Still other coagents are possible. For example, the coagent could include stem cells, with the resulting fibrin biopolymer serving as a scaffold for the stem cells. In a further embodiment, the powder 26 that includes the coagent could be combined with the contents of one or more of the compartments 21, 23, or 27, and thus only three compartments 21, 23, 27 would be present. In a still further embodiment, one or more of the coagents could be added to the compartments 21, 23, 27 and one or more of the other coagents would be present in the compartment 25. For example, an antibiotic (or another antibacterial agent, antifungal agent, or both) could be present in the compartments 21, 23, 27 to prevent bacterial or fungal growth while a protein coagent, such as ALT-C, with or without additional antibiotics, could be present in the compartment 25. In a still further embodiment, the powder 26 could be omitted, resulting in only three compartments being present in the device 10.

In a further embodiment, to protect the serinoprotease or a coagent (such as ALT-C protein) from degradation, the serinoprotease or the coagent could be microencapsulated in liposomes while stored in the respective compartments 23, 25. Liposomes are spherical vesicles having at least one lipid layer that can be created by a disrupting a biological membrane (such as by sonication). The liposomes can continue to encapsulate the serinoprotease, the coagent, or both, until the powders 24, 26, or both are mixed with contents of other compartments (such as the diluent 28 or the powder 22) that would include a substance that would cause a release of the contents of the liposomes (such as a detergent that would not denature the proteins encountered or interfere with the polymerization reaction).

The amounts of the fibrinogen, serinoprotease, the diluent, and the coagent in the compartments 21, 23, 25, 27 depends on how much fibrin biopolymer is necessary to be applied from the device 10. For example, in over-the-counter version sold for treatment of superficial skin damage, such as minor burns and cuts, the amounts of the components necessary to make the fibrin biopolymer could be less than in a version of the device 10 used by medical personnel at trauma centers where extensive skin damage that needs to be covered from a single device 10 can be expected. In one embodiment, once the powders 22 and 24 are dissolved within the diluent 28, the ratio of the concentrations of the powder 24 (the powder including the serinoprotease) to the powder 22 (the powder including the fibrinogen) to the calcium within the diluent 28 is 0.4:1.0:0.6. Other ratios are possible. The concentration of the coagent would depend on the kind of coagent used to create the fibrin biopolymer.

As mentioned above, once the seal 18 is broken, the contents of the compartments 21, 23, 25, and 27 enter a mixing chamber 19 seen with reference to FIGS. 3 and 6. FIG. 6 shows a horizontal cross-sectional view of the device 10 at the level marked as D-D on FIG. 1 in accordance with one embodiment. As can be seen with reference to FIGS. 2 and 6, the mixing chamber 19 includes curved, sloping walls that converge to define an opening 20 at the bottom of the chamber 19. The sloping of the walls of the chamber 19 slows down the rate at which the contents expelled from the compartments 21, 23, 25, 27 reach the opening 20, allowing the mixing of the contents expelled from different chambers to begin within the chamber 19. Further, additional mixing can occur upon movement of the device following the press of the button 12, either voluntary (such as to reposition the device 10) or involuntary (due to involuntary movement of the hand of the user holding the device caused by the effort of pressing the button 12).

The opening 20 is aligned and connected with the hollow portion of the tube 14 formed at the end of the housing 13, and thus the polymerizing mixture 36 that forms within the mixing chamber 19 travels (under the force of gravity) through the opening 20 into the tube and escapes the device through the distal end of the tube 14. Thus, a user can precisely apply the polymerizing mixture 35 (which will turn into the fibrin polymer once the polymerization is complete) to a target area by holding the tip of the tube 14 over the target area.

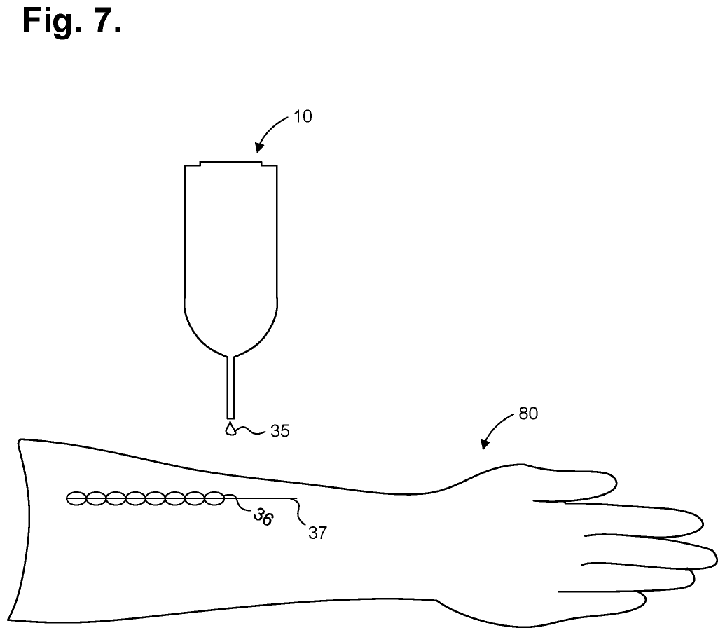

FIG. 7 is a diagram, provided for purposes of illustration, showing use of the device 10 for covering a wound 37 with the fibrin polymer 36. The wound 37 shown with reference to FIG. 7 is a long gash on the arm 80 of the patient (though applying the fibrin biopolymer to other kinds of wounds is also possible). After the button 12 has been depressed, drops 35 of the mixture in which the polymerization reaction has started begin dripping from the end of the tube 14. After the drops 35 land on the wound 37, they complete the polymerization to form a thin transparent layer of the fibrin biopolymer 36 over the wound 37. As the device 10 is moved along the wound 81, the drops 35 cover the entire length of the wound 37 and polymerize into the fibrin biopolymer 36 layer along the entire length of wound 37.

While in the description above, the device 10 has been described as being used for treatment of wounds, the device 10 also has a hemostatic surgical use, with the fibrin biopolymer dispensed using the device 10 being used to stop bleeding during surgery, seal a surgical cut, or other surgical use.

Also, while in the description above, the device 10 is described as applying the polymerizing mixture from a distance, when the tube 14 is a needle, the needle could also be inserted through the skin (or a mucous membrane) to deliver the polymerizing mixture 35 (and consequently the fibrin biopolymer 36) under into the site of the tumor, such as under the skin of the patient or on an internal organ of the patient. For example, if the patient has a cancerous tumor, the needle could be inserted into the skin and the polymerizing mixture could be inserted into the tumor or adjacently to the tumor. Any anti-cancer drugs (or proteins, such as the ALT-C or the lectin protein from Aplysia dactylomela) present as a coagent in the fibrin biopolymer 36 placed under the skin would then slowly be released (through osmosis or another mechanism of action) into the tumor. Such injection allows to avoid providing the anti-cancer medication (which often have high toxicity) via the oral route, thus reducing the overall toxicity of the medication to which the patient is exposed and increasing the portion of the anti-cancer medication that is delivered directly to the tumor. Other coagents could similarly be delivered directly to a desired location through an under-the-skin injection.

As shown with reference to FIGS. 3-6, which show portions of the device 10 in cross-section are shown with cross-hatching indicative that they are made of plastic, the device 10 can be made of at least in part (such as at least a portion of the tube 14) of metal. Alternatively, or in combination with plastic, metal, or both, other materials that do not react with the contents of the compartments 21, 23, 25, 27 or the fibrin biopolymer could be used in the device, such as glass. Further, while a particular mechanism of releasing the contents of the compartments 21, 23, 25, 27 into the mixing chamber 19 has been described, other mechanisms known in the art could be applied. For example, one or both of the seals 15 and 18 could be replaced with one or more flaps that pivot when enough force is applied to them. Thus, the one or more flaps replacing the seal 16 could pivot away when enough force the button 12 is pressed to allow the plungers 15 to enter the compartments 21, 23, 25, and 27 and the one or more flaps replacing the seals 18 could pivot away under the force applied by the contents of the compartments 21, 23, 25, and 27 when the contents are being pressured by the plungers, thus allowing the contents to enter the mixing chamber 19. Still other mechanisms are possible.

While the above mechanism allows for near-simultaneous release of all of the contents of all the compartments into the mixing chamber 19, in a further embodiment, a mechanism allowing for dosed release of the contents could be implemented.

The device 10 allows controlled, pinpoint application of the fibrin biopolymer desired locations (over which the tube 14 can be positioned). Sometimes, a wound area can be either too large for treatment using the contents of a single device 10, or distributed widely enough throughout the patient's body that covering all of the wound surface using a single device 10 before polymerization of the fibrin biopolymer is complete may be difficult. A device for applying substances that when mixed form a fibrin biopolymer described below provides additional control over the polymerization by allowing to deposit onto the skin components for creating the fibrin biopolymer in the form of foam or gel and to initiate the polymerization by mixing the components (such as with the user's hand) only when the user is ready.

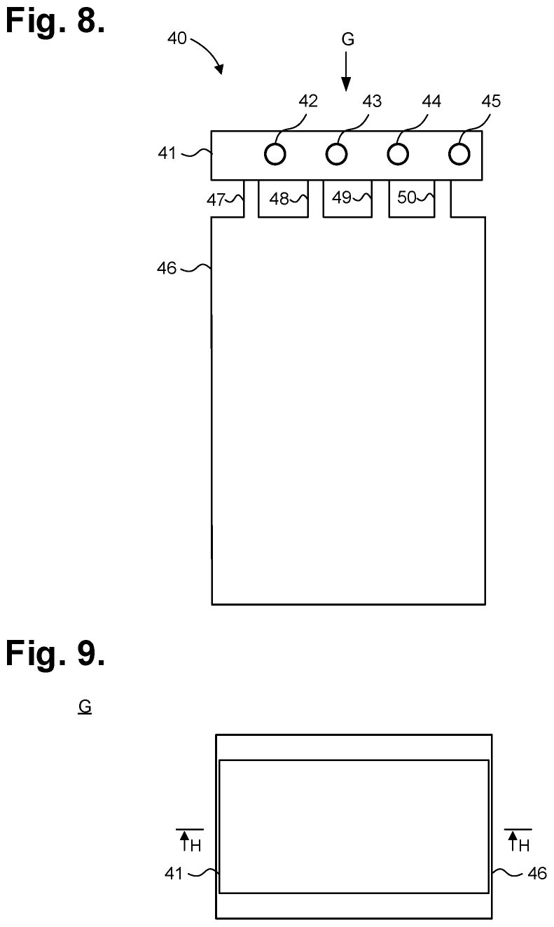

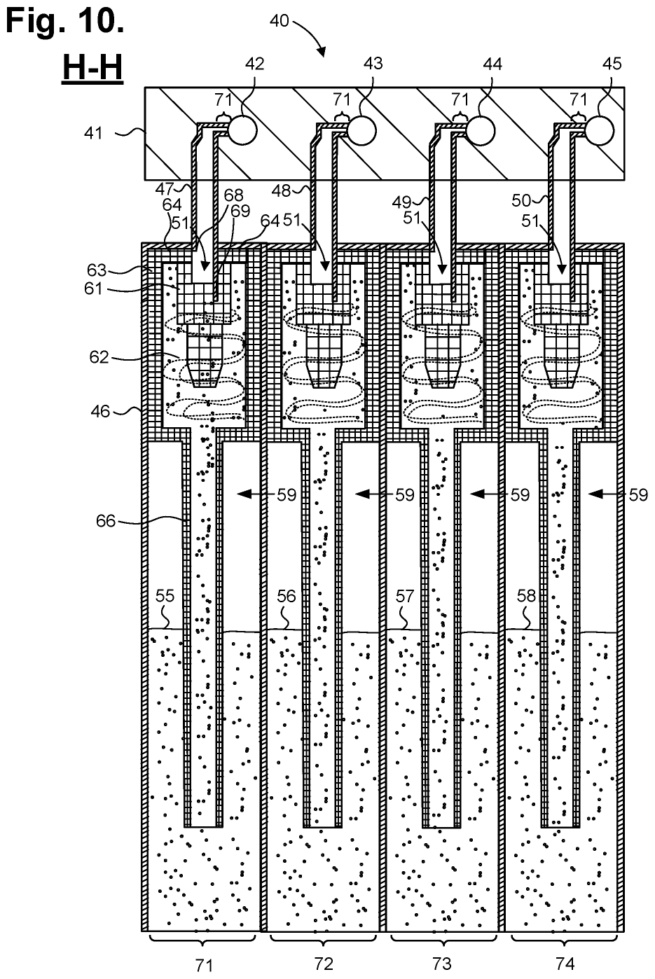

FIG. 8 is diagram showing a device 40 for fibrin-biopolymer-forming substance application in accordance with one embodiment. The device 40 includes a housing 46 and an actuator 41 connected to the housing 46 by hollow rods 47-50, which act as conduits through which formulations from the compartments within the housing (described below) can be provided into the actuator, with the formulations being expelled from the actuator via the openings (also referred to as orifices) 42-45. While the openings 42-45 are shown as offset from the hollow rods 47-50, in a further embodiment, the openings 42-45 could be located on the same level as the hollow rods 47-50. In one embodiment, the hollow rods 47-50 can be cylindrical, though other shapes of the hollow rods 47-50 are also possible. FIG. 9 is a diagram showing a top view of the device 40 of FIG. 8 in accordance with one embodiment. The top view is shown from the perspective marked as G in FIG. 8. As can be seen with reference to FIG. 9, the housing 46 and the actuator 41 can have a rectangular shape, though other shapes, including circular and oval shapes, are also possible. Still other shapes are possible. Further, while the top surface of the actuator 41 is shown as smaller than the top surface of the housing 46, in a further embodiment, other size proportions are possible.

Internally, the housing 46 includes compartments where components necessary for formation of a fibrin biopolymer are stored and which are released using actuation of a valve-based mechanism. FIG. 10 is a diagram showing a vertical cross-section of the device 40 of FIG. 9 at level marked H-H when the valves 51 are in closed positions. In one embodiment, the housing 46 includes multiple compartments s 71-74 separated from each other by partitions within the housing 46. The compartments 71-74 store formulations 55-58 that include components for formation of a fibrin biopolymer (with each compartment 71-74 storing one of the formulations). While the formulations 55-58 are shown as including black dots, the black dots are shown for the purposes of showing the movements of the formulations and do not necessarily represent the physical appearance of the formulations 55-58.

Each of the formulations 55-58 includes a component to be used for formation of the biopolymer. Thus, formulation 55 stored in the compartment 71 includes fibrinogen molecules. The fibrinogen can come as part of a fibrinogen-rich cryoprecipitate (insoluble, cold-precipitated fraction of frozen fresh plasma). The cryoprecipitate can be extracted from large animals through techniques known in the art, such as from bovine animals (such as buffalos), though other sources of the cryoprecipitate are possible. In one embodiment, the cryoprecipitate can include in addition to the fibrinogen factor VIII, factor V, and von Willebrand factor (such as in the case of a bubaline cryoprecipitate described in detail by the Ferreira publication cited above and whose disclosure is incorporated by reference). In a further embodiments, other components of the cryoprecipitate are possible. In a further embodiment, the fibrinogen in the formulation 55 is recombinantly produced in bacteria, yeast (or other fungi), mammalian cell culture, or other cell culture, and purified through chromatographic (such as high performance liquid chromatography or fast performance liquid chromatography) and other protein purification techniques known in the art. The recombinantly produced fibrinogen can be a bovine fibrinogen (such as from a buffalo fibrinogen), though other kinds of fibrinogen can also be made recombinantly. When a bovine or other non-human fibrinogen is used to make a fibrin biopolymer applied to a human, such fibrin biopolymer is considered heterologous.

The formulation 56 in the compartment 72 includes serinoprotease molecules capable of cleaving the fibrinogen in the formulation 55 into monomers necessary for creation of a fibrin biopolymer. Such serinoprotease can be gyroxine extracted from the snake Crotalus durissus terrificus described in detail by the Ferreira publication cited above and whose disclosure is incorporated by reference. Alternatively, the gyroxine can be produced recombinantly in bacteria, yeast (or other fungi), mammalian cell culture, or other cell culture, and purified using chromatographic (such as high performance liquid chromatography or fast performance liquid chromatography) and other protein purification techniques known in the art. In a further embodiment, the serinoprotease (a gyroxine or a similar serinoprotease) could be derived, either through extraction or through recombinant production (as described above), from other snakes, or other kinds of animals, such when the serionoprotease is a thrombin-like enzyme gyroxin B1.3 of the melon fruit fly Zeugodacus cucurbitae. Still other ways to obtain the serinoprotease are possible.

The formulation 57 in the compartment 73 includes calcium molecules, which are necessary for the fibrin monomers to polymerize into the fibrin biopolymer at a rate useful for practical applications, being a cofactor that catalyzes the polymerization reaction. The calcium molecules can be added to the formulation 73 as part of calcium chloride, calcium carbonate, or calcium phosphate, though dissolving calcium-containing molecules also possible. In a still further embodiment, another cofactor instead of calcium could be part of the formulation. As further described below, the formulations are expelled from the device 40 at an equal rate, and the concentration of calcium in the formulation 57 is such that the concentration of calcium ions within the a mixture formed from all four formulations 55-58 is 20 mM to 30 mM, though other concentrations are also possible. For example, with four formulations 55-58 being expelled from the device 40 at an equal rate, the initial concentration of calcium within the formulation 57 would be between 80 mM and 30 mM. In a further embodiment, if the number of formulations is not four, the concentration of calcium (or another catalyst) within the formulation 57 would be different. Similarly to what is described with respect to device 10, increasing or decreasing the calcium concentration outside of the 20 mM to 30 mM could also be used to control the rate of the polymerization once the formulations 55-58 are mixed.

Finally, the formulation 58 in compartment 74 stores includes one or more coagent molecules that when integrated into the fibrin biopolymer promote desired objectives, such as wound healing. For example, such coagent can include an antibiotic to prevent bacterial growth under the layer of fibrin biopolymer applied to a patient's skin. Other drugs could also be coagents. For example, one or more a coagent could be an anti-cancer drug, though other kinds of drugs are also possible. Alternatively or in addition to the drug, the coagent can be a lyophilized (or otherwise in powder form) protein. For example, such coagent could be Alternagin-C (ALT-C), an ECD-disintegrin-like protein from Bothrops alternatus snake venom shows antiangiogenic activity at concentrations higher than 100 nM. As angiogenesis, formation of new blood vessels, is crucial for tumor development, adding ALT-C (or another protein from the disintegrin family) as a coagent can be used during anti-cancer treatment. Other properties of ALT-C (or other disintegrin proteins) could be utilized for other kinds of treatments, such as in wound treatment. Still other kinds of proteins are also possible as a coagent. Still other coagents are possible. In a further embodiment, the coagent could be added to one or more of the formulations 55-57, and the formulation 58 would be omitted from the device 40. In that case, the compartment 74 would also be omitted from the device 40, and the device 40 would have only have three compartments 71-73. In a still further embodiment, one or more of the coagents could be added to the formulations 55-58 in one of the compartments 71-73 and one or more of the other coagents would be present in the formulation 58 in the compartment 74. For example, an antibiotic (or another antibacterial agent, antifungal agent, or both) could be present in the formulations 55-57 to prevent bacterial or fungal growth while a protein coagent, such as ALT-C, with or without additional antibiotics, could be present in the formulation 58 in the compartment 74. In a still further embodiment, coagents could be omitted entirely from the device 40, and thus only three compartments 71-73 would be present.

In a further embodiment, to protect the serinoprotease or a coagent (such as ALT-C protein) from degradation, the serinoprotease or the coagent could be microencapsulated in liposomes before being mixed with their respective formulations 56, 58. Liposomes are spherical vesicles having at least one lipid layer that can be created by a disrupting a biological membrane (such as by sonication). The liposomes can continue to encapsulate the serinoprotease, the coagent, or both, until the formulations 56, 58 or both are mixed with contents of other compartments (such as the formulations 55 or 57) that would include a substance that would cause a release of the contents of the liposomes (such as a detergent that would not denature the proteins encountered or interfere with the polymerization reaction).

In addition to including a substance (fibrinogen, serinoprotease, calcium (or another essential ion that could be used as a cofactor for the polymerization reaction), or one or more coagents) to be used directly in the formation of a fibrin polymer (also referred to as “biopolymer formation substances” in the description below), each of the formulations 55-58 includes one or more components that make up a “carrier” for the biopolymer formation substances in that formulation 55-58. In one embodiment, such carrier can be a substance (such as an emulsion) that is dispensed as a foam from the device 40 using one or more propellants described below, with a biopolymer formation substance being part of the foam-forming substance. For example, in addition to a biopolymer formation substance, such foam-forming substance can include water and one or more components commonly used to make foams that come in contact with human skin, such as stearic acid, myristic acid, potassium hydroxide, coconut acid, glycerin, triethanolamine, and sodium hydroxide, though other components are also possible. The ratios of the components in the foam-forming substance necessary to create the foam suitable for contact with human skin are known in the art, such as described in U.S. Pat. No. 4,111,827, issued Sep. 5, 1978 to Thompson et al. and U.S. Pat. No. 5,902,779, issued May 11, 1999, to Cormier et al., the disclosures of which are incorporated by reference, though still other components and their ratios are possible.

Alternatively to foam-forming substance, the carrier in each formulations 55-58 can be a soft gel suitable for contact with human skin within which a biopolymer formation substance is included. In one embodiment, the gel can be a post-foaming shaving gel, such as one whose composition is described in U.S. Pat. No. 5,858,343, issued Jan. 12, 1999, to Thomas J. Szymczak, the disclosure of which is incorporated by reference, though other post-foaming shaving gels known in the art could be used. While a post-foaming shaving gel is not dispensed as a foam, when rubbed against a person's skin, such gel tends to form foam, which can promote mixing of the biopolymer formation substances from different formulations 55-58 when the formulations 55-58 are mixed together on a patient's skin as described below. In a further embodiment, the soft gel could be without foaming properties and be made through a mixing of water, a biopolymer formation substance, and one or more gelling agents known in the art, such as natural gums, starches, pectins, agar-agar, gelatin, and other gelling agent, with the gelling agents being added using concentrations known in the art to create a gel of a desired viscosity. Still other kinds of soft gels are possible. As viscosity of substances that form foams when dispensed and of gels of different compositions can be different, the same carrier is used in all of the formulations 55-58 to ensure that all of the formulations have the same or substantially the same viscosity and thus escape from the device 40 at the same or substantially the same rate. The carrier that is used is more viscous than water, which, as described below, allows for greater control of how the manner of the fibrin biopolymer formation.

The hollow rods 47-50 are integrated into the actuator 41 and each of the hollow rods 47-50 are connected by a passage 71 to one of the orifices 42-45. Each of the compartments further includes a valve 51 connected to one of the rods 47-50 and that takes a closed position (in which the formulation 55-58 in the compartment 71-74 in which that valve 51 is located is prevented from traveling via one of the hollow rods 47-50 to one of the openings 42-45) and an open position (in which the formulation 55-58 in the compartment 71-74 in which that valve 51 is located has a path to travel via one of the hollow rods 47-50 to one of the openings 42-45). The valves 51 can be secured within the housing using a plurality of techniques known in the art, such as using a mountain cup (not shown) that would fit around the hollow rods 47-50, such as a cup described in U.S. Pat. No. 4,111,339, issued Sep. 5, 1978 to Schmidt, the disclosure of which is incorporated by reference, though other ways to secure the valves 51 within the housing 46 are possible.

While a particular configuration of the valves 51 is described below, other configurations of the valves 51 could also be used, such as those described in U.S. Pat. No. 3,391,834, issued Jul. 9, 1968, to Focht, the disclosure of which is incorporated by reference, and U.S. Pat. No. 2,881,908, issued Apr. 14, 1959, to Germain, the disclosure of which is incorporated by reference, though many other valve designs could be used.

While the valves 51 in all of the compartments 71-74 as well as the hollow rods 47-50 connected to the valves 51 and the openings 42-50 connected to those hollow rods 47-50 are identical, for the sake of clarity of the drawings, some of the elements making up the valves 51 and the hollow rods 47-50 have been labeled in only one of the four valves 51 and the hollow rods 47-50.

Each valve 51 includes a valve body 63 that defines a hollow, substantially cuboid (though other shapes are possible) that is fixedly attached within an interior of one the compartments 71-74 and that defines two openings 52, 53. A gasket 64 acts as a top (with reference to the orientation seen with reference to FIG. 10) wall of the valve body 53 and surrounds the opening 53.

While the gasket 64 is shown with reference to FIG. 10 as being an integral part of the valve body 63, in a further embodiment, the gasket 64 could be made of a different material (such as a different type of plastic) than the rest of the valve body 63 or could be fixedly attached to the rest of the valve body 63 (such as through matching mechanical interfaces) rather than being an integral part of the body.

A dip tube 66 that is formed around the opening 53 on the valve body 63 of each of the valves 51 and extends into one of the formulations 55-58, providing the formulation 55-58 into which the dip tube 66 extends a path into the inside of the valve body 63 on which that tube 66 is formed.

The valve 51 further spring a spring cup 61 that is set within the valve body 63. One end of the spring cup 61 is connected set within a spring 62 that is set at the bottom of the valve body 63 above the opening 53 (in the orientation shown with reference to FIG. 10). The other end of the spring cup 61 (the end opposite to the end set within the spring 62) has an surface that, when the valve 51 is in the closed position, is pushed against the gasket 64 in a way that creates a seal separating the opening 52 from the formulations 55-58, thus cutting off a path for one of the formulations 55-58 to enter one of the hollow rods 47-50.

The end of the spring cup 61 that is in contact with the gasket 64 in the closed position of the valve is also connected to one of the rods 47-50. The ends of the rods 47-50 that reach into the device housing 46 are not of a uniform length on all sides; thus, one portion 68 of the end is shorter than other portions and does not touch the spring cup 61 (but is of a sufficient length to reach the housing 46, thus preventing any external objects from entering into the housing 46 regardless of the position of the valve) while at least one other portion 69 is longer and is in contact with the spring cup 62 (such as due to being fixedly attached to the cup 61). The disparity in the length of portions 67 of the rods 47-50 creates an opening through which one of the formulations 55-58 can enter into the interior of the hollow rods when the valve 51 switches into the open position.

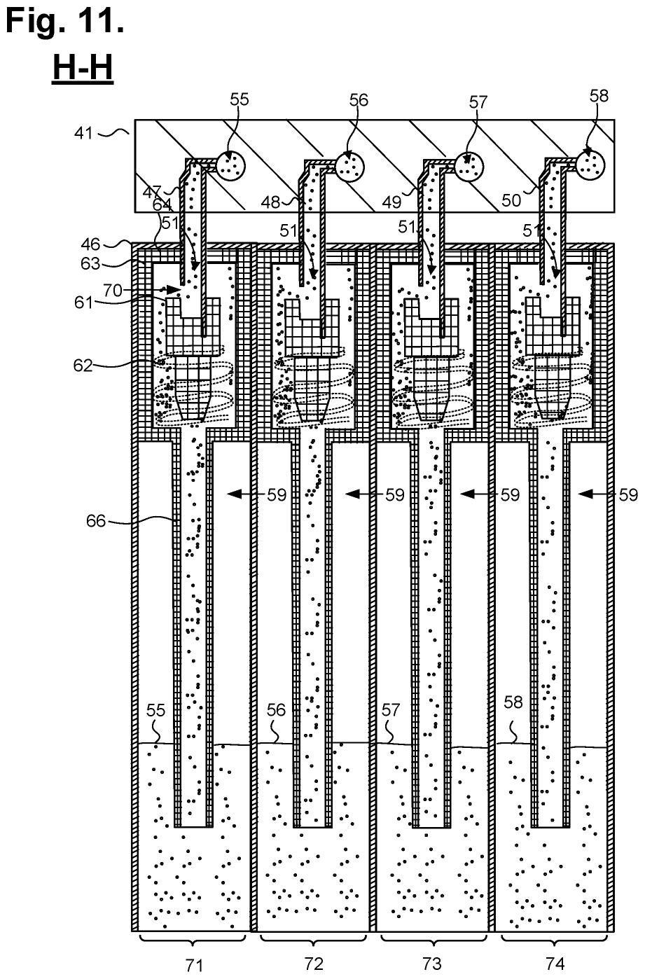

The switching of the valve 51 from the closed position is accomplished by pressing on the actuator 41. FIG. 11 is a diagram showing the vertical cross-section of the device 40 of FIG. 9 at level marked H-H after the valves 51 have shifted into an open position. When downward (relative to the orientation shown with reference to FIG. 11) pressure is applied to the actuator 46 by a user of the device, the portions 69 of the hollow rods 47-50 that are in contact with the spring cups 61 push against the spring cups 61 downward, compressing the spring 62 and breaking the seal between the spring cup 61 and the gasket 64.

Because the spring cup 61 and the gasket 64 are no longer in contact, the formulations 55-58 have a path to enter the interior of the hollow rods 47-50 into via the openings 70 created due to the ends inside the housing 46 of the hollow rods 47-50 not being of a uniform length on all sides. In a further embodiment, instead of the opening 70 being present due to due to the ends 67 of the hollow rods 47-50 not being of a uniform length on all sides, all sides of the end 67 could be of a uniform length, but the opening 70 could be an aperture within the end 67 that is positioned within the housing 46 (being in contact with the spring cup 61) both in the open and the closed positions of the valve; when the pressure is applied to the actuator 41, the aperture would be inside the portion of the valve body 63 into which one of the formulations 55-58 can enter in the absence of the seal between the spring cap 61 and the gasket 64. Once the pressure is removed from the actuator 41, springs 62 push the spring cups 61 back in contact with the gasket 64 (which in turn pushes the actuator 41 to the original position via the portions of the hollow rods 47-50 attached to the spring cups 61). In the embodiment where the formulations 55-58 would enter through the aperture, upon removal of the pressure, the aperture would be pushed shifted into a position where the aperture is blocked by the gasket 64 (with the actuator 41 returning to the original position due to the push of the spring 62). The opening and closing of the valves 51 by applying pressure to the actuator 41 allows to dispense a desired amount of the formulations 55-58 and to stop and restart the dispensing as needed.

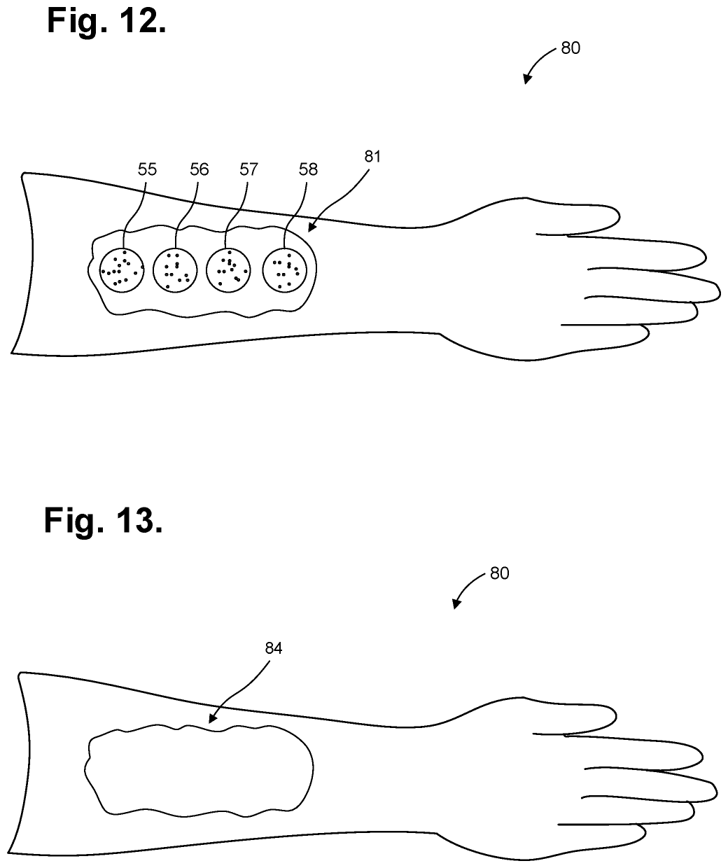

Returning to FIG. 10, the formulations 55-58 travel using the path due to a pressure differential that exists between the interior of the compartments 71-74 and the external environment. Before the valves 51 are opened to dispense the formulations 55-58 for a first time, the pressure inside the compartments 71-74 can be between 3 bars-10 bars at room temperature, though other values are also possible. In addition to one of the formulations 55-58, each of the compartments 71-73 includes at least one pressurized propellant, which can be a single one or a mixture of gases, including liquefiable gases (gases that take a liquid form when under a high pressure even when above the gases' boiling point temperature) or compressible gases. Such at least one pressurized propellant 59 can include one or more of nitrous oxide, carbon dioxide, a hydrocarbon (such as propane, n-butane, or isobutane), one or more hydrofluoroalkanes (such as 1,1,1,2,-tetrafluoroethane or 1,1,1,2,3,3,3-heptafluoropropane), or one or more hydrofluoroolefins, though still other propellants are possible. During manufacturing of the device 40, the propellants 59 are added to the compartments 71-74 under a high pressure (such as through the valve 51 in the open position). When the at least one propellant 59 includes a compressible gas such as nitrous oxide, at least a majority of the compressible gas would end up at the upper (in the orientation shown with reference to FIGS. 10 and 11) portion of the compartments 71-74 and would exert pressure onto the formulations 55-58 that would drive a portion of the formulations 55-58 into the dip tubes and into the interior of the valves 51. However, while the valves 51 remains in the closed position, the formulations 55-58 can't travel through the opening 52 due to the seal between the gaskets 64 and the spring cup 61. When the valves 51 shifts into the open positions, as shown with reference to FIG. 11, the pressure from the compressed gas drives the formulations 55-58 into the interior of the hollow rods 47-50 through the openings 70, with the formulations 55-58 through the hollow rods and into the openings 42-45 in the actuator 41. The formulations 55-58 then exit through the openings 42-45, and when the actuator 41 is positioned near a wound that needs to be treated, are applied to that wound, as further described below with reference to FIGS. 12-13.

Similarly, when the propellant 59 includes a liquefiable gas, such as isobutane, during manufacturing, the liquid propellants is pushed into the compartments 71-74 in liquid form under a high pressure (being placed on top of the formulations 55-58 when considering the orientation shown with reference to FIGS. 10 and 11), and at least a majority of the liquid propellant 59 remains a liquid while the valve remains closed. The opening of the valves 51 decreases the pressure inside the compartment 71-74 to an extent that allows the liquid propellant to start boiling and form a layer of gas at the top portion of the compartments 71-74, which pushes the formulations 55-58 and some of the propellant in liquid form to travel up through the dip tubes into the valve body 63, into the interior of the hollow rods 47-50, and into and through the openings 42-45, thus being expelled from device 40.

As mentioned above, the at least one propellant can include at least one compressible gas, at least one liquefiable gas, or a mixture of compressible and liquefiable gases. For the purposes of illustration, in the closed position, the formulations 55-58 is shown as being inside the valve body 63, though other positions are also possible.

The components of the device 40 are made of a material that can withstand the pressure within the compartments 71-74. Thus, the housing 46 and the actuator 46 can be made of tinplate (steel with a layer of tin) or aluminum, though other non-biologically reactive materials are also possible. The valve 51 can be made of a pressure-resistant plastic, though other materials are also possible. With reference to FIGS. 10 and 11, the cross hatching is used on the sliced exterior surfaces of the valve 51 to indicate those materials being made of plastic. Similarly, angled lines are used to indicate metal on the housing 41, the hollow rods 51, and the actuator 41. In a further embodiment, other materials could be used for those components.

Pressing the actuator 41 creates four streams of the formulations 55-58 being dispensed from the four openings 42-45. As the openings are set apart from each other, the formulations are deposited on a desired surface (such as a skin of a patient) some distance from each other, and unless other the actuator 41 remains depressed for an extended period of time near the same spot on the skin, the dispensed formulations do not initially mix with each other, as can be seen with reference to FIG. 12. FIG. 12 is a diagram showing an example of the formulations 55-58 deposited by the device 40 of FIG. 8 on a wound 81 on a patient's arm 80. The formulations 55-58 are some distance from each other and do not cover the entire surface of the wound 81. Further, due to the higher viscosity of the formulations 55-58 (when compared to water), the formulations 55-58 do not easily shift from their positions on the arm 80 due to gravity or accidental movements of the arm 80. Thus, as the polymerization reaction does not begin immediately upon the application of the formulations 55-58, a person using the device 40 has sufficient time to apply the desired amount of the formulations to desired area (or areas) on the skin that needs to be treated.

Once the desired amounts of the formulations have been applied using the device 40 to the skin, the patient (or another person applying the formulations 55-58 using the device) can initiate the polymerization reaction by mixing the applied formulations 55-58 either with his or her hand or with another object. While mixing the formulations 55-58, the person has a chance to cover with the mixture the entire area that needs to be covered with the fibrin biopolymer, as can be seen with reference to FIG. 13. FIG. 13 is a diagram showing an example of the fibrin biopolymer 84 formed on top of the wound 81 shown with reference to FIG. 12 following the mixing of the formulations 55-58 applied to the wound with the device 40. While the dispensed formulations 55-58 did not cover the entire surface of the wound 81, the thin layer of the fibrin biopolymer 81 that results from the mixing of the formulations does cover the entire wound 81, promoting heating in that wound 81. While the formulations 55-58 shown with reference to FIG. 12 were shown as applied to the skin at only a single spot, by moving the device 40 along the skin of the patient while pressing the actuator 41, the user of the device can apply the formulations along an elongated skin area, and to initiate the polymerization reaction of each stretch of the applied formulations 55-58 only when ready. Thus, the device 40 provides increased control over how and when the polymerization that leads to the formation of the fibrin begins. Further, the ease of use of the device 40 makes the device 40 usable for medical professionals and for users who bought the device over-the-counter.

When the formulations 55-58 are mixed in equal proportions, the ratio of the concentrations of the serinoprotease to the concentration of the fibrinogen to the concentration of calcium in the resulting mixture are 0.4:1.0:0.6, though other concentration ratios are also possible in a further embodiment. The concentration of any coagents would depend on the kind of a coagent used.

While in the description above, the device 40 is described as being used for treating external wounds, in a further embodiment, with appropriate formulations 55-58, the device 40 could have a hemostatic surgery use for stopping bleeding and sealing cuts as described above.

While the invention has been particularly shown and described as referenced to the embodiments thereof, those skilled in the art will understand that the foregoing and other changes in form and detail may be made therein without departing from the spirit and scope.

What is claimed is:

1. A device for fibrin-biopolymer-forming substance application, comprising:
a housing;
a plurality of compartments formed within the housing, contents of each of the compartments comprising one or more propellants, the plurality of the compartments comprising:
a fibrinogen compartment whose contents further comprise a formulation more viscous than water and comprising fibrinogen;
a serinoprotease compartment whose contents further comprise a further formulation more viscous than water and comprising a serinoprotease, wherein the serinoprotease cleaves the fibrinogen into fibrin monomers when combined with the fibrinogen; and
a coagent compartment whose contents comprise an additional formulation more viscous than water and comprising a coagent, wherein the fibrin monomers polymerize into a fibrin biopolymer in the presence of the coagent;
a plurality of valves, each of the valves integrated into one of the compartments and adapted to take a closed position and an open position;
a plurality of at least partially hollow conduits, each of the conduits comprising a first end that is at least partially interfaced to one of the valves, the first end comprising a first opening, each of the conduits further comprising a second end comprising a second opening that is interfaced to one of a plurality of orifices within an actuator;
the actuator interfaced to all of the conduits and configured to apply pressure to all of the valves via the conduits upon being pressed by a user, wherein the pressure shifts the valves from the closed position into the open position and exposes the first opening of each of the conduits to the contents of the compartments, and wherein at least part of the contents of each of the compartments is propelled by the one or more propellants via the first openings of the conduits to flow via the conduits out of the second openings and out of the orifices of the actuator while the valves remain in the open position, and wherein the fibrinogen formulation, the serinoprotease formulation, and the coagent formulation form the fibrin polymer when mixed outside of the actuator.

2. A device according to claim 1, wherein the plurality of the compartments further comprises a coagent compartment whose contents further comprise a still further formulation comprising one or more coagents, wherein at least a portion of the one or more coagents is integrated within the fibrin biopolymer upon a completion of the polymerization of at least the portion of the fibrinogen into the fibrin biopolymer.

3. A device according to claim 2, wherein the one or more coagents comprise one or more of a drug, a protein, a stem cells, an antibacterial agent, and an antifungal agent.

4. A device according to claim 3, wherein the protein comprises Alternagin-C protein and the drug comprises at least one of an antibiotic and an anti-cancer drug.

5. A device according to claim 1, wherein the more of the fibrinogen formulation, the serinoprotease formulation, and the cofactor formulation further comprise one or more coagents, and wherein at least a portion of the coagents is integrated into the biopolymer upon a completion of the polymerization of at least the portion of the fibrin monomers into the fibrin biopolymer.

6. A device according to claim 5, wherein the one or more coagents comprises one or more of a drug, a protein, a stem cells, an antibacterial agent, and an antifungal agent.

7. A device according to claim 6, wherein the protein comprises Alternagin-C protein and the drug comprises at least one of an antibiotic and an anti-cancer drug.

8. A device according to claim 1, wherein the cofactor comprises calcium.

9. A device according to claim 8, wherein the calcium is added to the cofactor formulation as one or more of calcium chloride, calcium carbonate, and calcium phosphate.

10. A device according to claim 1, wherein the serinoprotease is derived from one of a snake and a fly.

11. A device according to claim 1, wherein formulations do not come into contact with each other when flowing out of the orifices.

12. A device according to claim 1, wherein each of the formulations comprises one of an emulsion that forms a foam when propelled by the one or more propellants and a gel.

13. A device according to claim 12, wherein the foam comprises one or more of water, stearic acid, myristic acid, potassium hydroxide, coconut acid, glycerin, triethanolamine, and sodium hydroxide.

14. A device according to claim 12, wherein the gel forms a further foam when rubbed against a skin of a patient.

15. A device according to claim 12, wherein the viscosity of all of the formulations is substantially the same.

16. A device according to claim 1, wherein the one or more propellants comprise one or more compressible gases and one or more liquifibiable gases.

17. A device according to claim 16, wherein the one or more propellants further comprises one or more of nitrous oxide, carbon dioxide, one or more hydrofluoroalkanes, one or more hydrofluoroolefins, and one or more hydrocarbons comprising one or more of propane, n-butane, and isobutane.

18. A device according to claim 1, wherein the fibrin biopolymer is heterologous to humans.

19. A device according to claim 1, wherein the fibrinogen formulation comprises a cryoprecipitate extracted from a bovine animal, the cryoprecipitate comprising the fibrinogen.

20. A device according to claim 1, wherein the fibrinogen and the serinoprotease are recombinantly produced.
